(19) United States
(12) Patent Application Publication
(10) Pub. No.: US 2005/0015122 A1
(43) Pub. Date: Jan. 20, 2005

(54) System and method for control of a subject's circadian cycle

(76) Inventors: Christopher Grey Mott, Vancouver (CA); Daniel Joseph Mollicone, Windsor (CA); Matthew Van Wollen, Vancouver (CA); Mihai Huzmezan, White Rock (CA)

Correspondence Address: OYEN, WIGGS, GREEN & MUTALA;480 - THE STATION, 601 WEST CORDOVA STREET, VANCOUVER, BC, V6B 1G1, CA

(21) Appl. No.: 10/859,172

(22) Filed: Jun. 3, 2004

Related U.S. Application Data
(60) Provisional application No. 60/475,529, filed on Jun. 3, 2003.

(51) Int. Cl. A61N 5/06

(52) U.S. Cl. 607/88

(57) ABSTRACT

Aspects of the invention provide systems and methods for controllably adjusting the circadian pacemaker cycle of a subject using light (or other stimulus) through application of model-based predictive control techniques. This approach allows the use of closed-loop feedback to compensate for modeling errors, unknown initial conditions and disturbances. It also allows an optimal level of light (or other stimulus) to be generated based on minimization of a cost function. The cost function may incorporate a term associated with tracking errors and a term associated with the amount of light used. The tracking function may be minimized subject to one or more constraints which may include a minimum and maximum amount of light (or other stimulus).

### RELATED APPLICATIONS

This application claims the benefit of the filing date of U.S. Patent Application No. 60/475529 filed 3 Jun. 2003.

### TECHNICAL FIELD

The invention relates to a system and method for modifying the circadian cycle of a subject. Particular embodiments of the invention involve providing stimulus to controllably adjust a subject's circadian cycle to track a reference trajectory.

### BACKGROUND

Biological organisms of many kinds exhibit cycles which manifest themselves in a variety of physiological and behavioral characteristics. The cycles may have periods of hours (ultradian), days (circadian), or longer intervals (infradian). Circadian cycles are commonly observed in humans, as is evidenced by fluctuations in core body temperature, hormone levels, alertness and cognitive performance, for example. Of particular interest are alertness and cognitive performance, as these characteristics are correlated to health, safety and productivity.

There are circumstances where it may be desirable to alter or otherwise control a circadian cycle of a subject. For example, it may be desirable to maximize the alertness or cognitive performance of a subject for particular periods of time during which they are required to perform important or dangerous tasks, such as when they are flying an aircraft or performing an operation.

The biological basis of circadian cycles in humans is believed to be an endogenous circadian pacemaker: a self-oscillating chemical process in the suprachiasmatic nucleus region of the brain with a period very near to 24 hours (see, for example, C. A. Czeisler, J. F. Duffy, T. L. Shanahan, E. N. Brown, J. F. Mitchell D. W. Rimmer, J. M. Ronda, E. J. Silva, J. S. Allan, J. S. Emens, D. J., Dijk, and R. E. Kronauer., “Stability, precision, and near-24-hour period of the human circadian pacemaker.” Science, Vol. 284, No.5423 (1999), pp. 2177-2181). External stimuli influence the endogenous circadian pacemaker cycle, and in turn, the various circadian cycles that it regulates. Light is a particularly effective stimulus for modifying the circadian pacemaker cycle of a human subject (see, for example, C. A. Czeisler, J. S. Allan, S. H. Strogatz, J. M. Ronda, R. Sanchez, C. D. Rios, W. O. Freitag, G. S. Richardson, and R. E. Kronauer, “Bright light resets the human circadian pacemaker independent of the timing of the sleep-wake cycle.” Science, Vol. 233, No. 4764, (August 1986), pp.667-671).

The effect of external stimuli (such as light) on the human circadian pacemaker cycle has led to a number of patents, including: U.S. Pat. Nos. 5,163,426; 5,167,228; 5,176,133; 5,304,212; and 5,545,192 (to Czeisler et al.); U.S. Pat. Nos. 5,343,121; and 5,589,741 (to Terman et al.); U.S. Pat. No. 6,350,275 (to Vreman). The techniques proposed by these patents have a variety of limitations which include, inter alia: slow or otherwise non-optimal shifting of the subject's circadian pacemaker cycle; lack of feedback based on measurement of parameter(s) indicative of the state of the subject's circadian pacemaker cycle; lack of mechanisms for incorporating limitations on available stimuli; lack of mechanisms for incorporating differences between individual subjects, modeling errors, unknown initial conditions and/or the introduction of disturbances, and lack of a continuous connection or dependence between assessment of the subject's circadian state and the modification of the subject's circadian pacemaker cycle.

There is a general desire to provide systems and methods for controllably adjusting a circadian cycle of a subject which overcome or ameliorate some of the aforementioned disadvantages of the prior art.

### SUMMARY OF THE INVENTION

A first aspect of the invention provides a method for controllably adjusting the circadian cycle of a subject using light (or other stimulus) through application of model-based predictive control techniques. This approach allows the use of closed-loop feedback to compensate for modeling errors, unknown initial conditions and disturbances. It also allows an optimal level of light (or other stimulus) to be generated based on minimization of a cost function. The cost function may incorporate a term associated with tracking errors and a term associated with the amount of light used. The tracking function may be minimized subject to one or more constraints which may include a minimum and maximum amount of light (or other stimulus) for example.

Another aspect of the invention provides a method for controllably adjusting a circadian cycle of a subject to track a reference trajectory. The method involves providing at least one model representative of a response of a circadian state of the subject to a stimulus control signal and generating an optimal stimulus control signal using model predictive control based on the model. Generating the optimal stimulus control signal comprises minimizing a cost function which may comprise a cost term which is a function of a tracking error and a cost term related to an amplitude of the stimulus control signal. Minimizing the cost function is subject to constraints which comprise minimum and maximum stimulus control signal levels.

Generating the optimal stimulus control signal may be based, at least in part, on a circadian state feedback signal. The circadian state feedback signal may be determined by modeling the response of the circadian state of the subject to the stimulus control signal. Modeling the response of the subject to the stimulus control signal may comprise using a mathematical model based on a Jewett-Kronauer model. The mathematical model may comprise a linearized version of the Jewett-Kronauer model. The circadian state feedback signal may be determined by estimating the circadian state of the subject based, at least in part, on one or more sensed parameters which relate to the physiology of the subject. The sensed parameters may include: heart rate, core body temperature, respiration, endocrine function levels, physical activity levels, blood pressure, blood oxygen concentration and/or skin temperature.

The stimulus control signal may comprises a light control signal and the method may comprise applying the light control signal to determine an intensity of one or more light sources.

The at least one model may comprise a response predictor subject model. Generating the optimal stimulus control signal may comprise predicting a free response of the circadian state of the subject starting at a current time and extending out to a control horizon time using the response predictor subject model. Predicting the free response may comprise assuming that a current value of the circadian state feedback signal represents an initial condition and that the light control signal remains constant between the current time and the control horizon time.

The at least one model may comprise a controller subject model. Generating the optimal stimulus control signal may comprise determining an optimal series of control moves starting at the current time and extending out to the control horizon time using the controller subject model. Determining the optimal series of control moves may be based, at least in part, on the free response of the circadian state of the subject, the reference trajectory and the circadian state feedback signal.

The response predictor subject model and/or the controller subject model may comprise a mathematical model defined by a plurality of differential equations. The mathematical model may be based on a Jewett-Kronauer model. The mathematical model may comprise a linearized version of the Jewett-Kronauer model.

Predicting the free response may be based, at least in part, on a light estimate signal representative of a light intensity experienced by the subject. Determining the optimal series of control moves may be based, at least in part, on the light estimate signal. The method may involve estimating the light intensity experienced by the subject using a model to obtain the light estimate signal. The method may involve sensing the light intensity experienced by the subject using at least one light sensor to obtain the light estimate signal.

The stimulus control signal may be provided in a domain of a driving input B of the Jewett-Kronauer model. The method may comprise converting the stimulus control signal from the domain of the driving input B to the light control signal in a light intensity domain I. The method may also comprise converting the minimum and maximum stimulus control signal levels from a light intensity domain I to a domain of a driving input B of the Jewett-Kronauer model.

The cost function may comprise a first weighting factor associated with the tracking error cost term and a second weighting factor associated with the stimulus control signal cost term. The first and second weighting factors may vary over a duration of the circadian cycle of the subject.

Another aspect of the invention provides a method for controllably adjusting a circadian cycle of a subject to track a reference trajectory. The method involves obtaining input information which comprises a signal representative of the reference trajectory, a feedback signal representative of a circadian state of the subject and one or more stimulus level constraints. The method also involves predicting a free response of the circadian state of the subject starting at a current time and extending out to a first future time using a response predictor subject model representative of a response of the circadian state of the subject to a stimulus. An optimal series of control moves is determined starting at the current time and extending out to second future time using a controller subject model representative of a response of the circadian state of the subject to a stimulus. The optimal series of control moves is based, at least in part, on the free response of the circadian state of the subject, the reference trajectory signal and the current circadian state feedback signal. The method also involves outputting a stimulus control signal comprising a current one of the series of control moves to one or more stimulus sources and applying the stimulus control signal to one or more stimulus sources to determine an intensity thereof.

Another aspect of the invention comprises a system for controllably adjusting a circadian cycle of a subject to track a reference trajectory. The system comprises one or more inputs for receiving the reference trajectory and for receiving constraints which comprise minimum and maximum stimulus control signal levels. A response predictor is connected to receive a feedback signal representative of a circadian state of the subject and is configured to predict a free response of the circadian state of the subject starting at a current time and extending out to a first future time using a response predictor subject model representative of a response of the circadian state of the subject to a stimulus. A control sequence generator is connected to receive the reference trajectory, the constraints, the feedback signal and the free response of the circadian state of the subject. The control sequence generator is configured to determine an optimal series of control moves starting at the current time and extending out to a second future time using a controller subject model representative of a response of the circadian state of the subject to a stimulus. The control sequence generator is connected to output a stimulus control signal to at least one stimulus source. The stimulus control signal comprises a current control move in the optimal series of control moves and the stimulus control signal determines an intensity of the at least one stimulus source.

Yet another aspect of the invention provides a system for controllably adjusting a circadian cycle of a subject to track a reference trajectory. The system comprises one or more inputs for receiving the reference trajectory and for receiving constraints which comprise minimum and maximum stimulus control signal levels. The system also comprises means for predicting a free response of a circadian state of the subject starting at a current time and extending out to a first future time based, at least in part, on a feedback signal representative of the circadian state of the subject and means for determining an optimal series of control moves starting at the current time and extending out to a second future time based, at least in part, on the reference trajectory, the constraints, the feedback signal and the free response of the circadian state of the subject. Means for applying a stimulus control signal to at least one stimulus source are also provided. The stimulus control signal comprises at least a portion of the optimal series of control moves and the stimulus control signal determines an intensity of the at least one stimulus source.

Still another aspect of the invention provides a method for altering a phase of a circadian cycle of a subject. The method comprises providing a controller subject model representing a response of a circadian state of the subject to a stimulus, receiving a reference circadian trajectory, which is phase shifted from a current circadian cycle of the subject, determining a series of stimulus control moves predicted by the controller subject model to result in the circadian cycle of the subject changing to track the reference circadian trajectory and applying at least a portion of the series of stimulus control moves to one or more stimulus sources. The one or more stimulus sources provide stimulus which is received by the subject. Determining the series of stimulus control moves comprises applying an optimization process using the controller subject model.

Further features and applications of specific embodiments of the invention are described below.

### BRIEF DESCRIPTION OF THE DRAWINGS

In drawings which depict non-limiting embodiments of the invention:

FIG. 1 is an exemplary graph depicting a response of a subject's circadian state to a pulse of light;

FIG. 2 is an exemplary graph depicting a comparison of circadian states predicted by a nonlinear model and circadian states predicted using a linear approximation of the nonlinear model;

FIG. 3 is a block diagram of a system for controllably adjusting a circadian cycle of a subject in accordance with a particular embodiment of the invention;

FIG. 4 is a schematic diagram of the light controller of the FIG. 3 system in accordance with a particular embodiment of the invention;

FIG. 5 shows a number of exemplary graphs which demonstrate how the FIG. 3 system may be used to controllably adjust the circadian pacemaker cycle of a subject to track a reference trajectory;

FIG. 6 shows a number of exemplary graphs which demonstrate how the FIG. 3 system may be used to controllably adjust the circadian pacemaker cycle of a subject to track a reference trajectory;

FIG. 7 is a block diagram of a system for controllably adjusting a circadian cycle of a subject in accordance with another embodiment of the invention; and

FIG. 8 shows a method for implementing the FIG. 3 or FIG. 7 system in accordance with a particular embodiment of the invention.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

Aspects of the present invention provide systems and methods for controlling the circadian pacemaker cycle of a subject to track a reference trajectory. Controlled amounts of light (or other stimulus) are provided to the subject in such a manner that the subject's circadian pacemaker cycle is caused to track the reference trajectory. In some embodiments, the control systems and methods make use of one or more models. Such models may empirically describe the response of a typical subject's circadian state to light or other stimulus. Feedback may be used to compensate for differences between individuals, modeling errors, unknown initial conditions and other disturbances.

The control systems and methods may incorporate one or more physiological sensors, which sense one or more parameters that are correlated to or otherwise indicate the subject's circadian state. Such parameter(s) may be used as feedback. In some embodiments, the control systems and methods comprise one or more stimulus sensors, which sense the amount light (or other stimulus) applied to the subject and which feed this information back to the controller.

In some embodiments, the systems and methods determine an “optimum” application of light (or other stimulus) to adjust the subject's circadian cycle to the desired reference trajectory. The “optimum” application of light may be achieved by substantially minimizing a cost function. In some embodiments, the cost function incorporates a term related to the tracking error and a term related to the amount of stimulus required. The optimization cost function may be minimized subject to physical constraints inherent in the system. Such constraints may include a minimum and maximum amount of light (or other stimulus).

In some embodiments, the models used to predict the subject's circadian state comprise nonlinear models. The systems and methods may comprise a variety of approximation techniques for transforming or otherwise reducing such nonlinear models to linear models.

In some embodiments, the reference trajectory is selected so that the subject is more alert in certain desired time periods.

Some embodiments of the invention make use of mathematical models which describe the response of a human circadian pacemaker state to light (or other stimulus). Such models may be empirically determined. In some embodiments, such models comprise a system of differential equations derived from the well known van der Pol equations. Particular embodiments of the invention make use of the model described in M. E. Jewett, D. B. Forger, and R. E. Kronauer, “Revised Limit Cycle Oscillator Model of Human Circadian Pacemaker.” Journal of Biological Rhythms, Vol. 14, No. 6 (1999), pp. 493-499, which is hereby incorporated herein by reference.

The Jewett-Kronauer model comprises a set of differential equations which describe the nonlinear effects of light on the circadian pacemaker cycle. In accordance with the Jewett-Kronauer model, the circadian state of a subject is described by a pair of circadian state variables (x, x_{c}). The circadian state variables (x, x_{c}) are mathematical constructs which represent the circadian state of a subject. According to the Jewett-Kronauer model, the response of the circadian state variables (x, x_{c}) to a driving input B is given by:

$$\begin{matrix}\dot{x} = \frac{π}{12} [ {x}_{c} + μ ( \frac{1}{3} x + \frac{4}{3} {x}^{3} - \frac{256}{105} {x}^{7} ) + B ] & ( 1 ) \\ \dot{{x}_{c}} = \frac{π}{12} {qBx}_{c} - [ ( \frac{24}{{τ}_{x} ( 0.99729 )} ) + kB ] x & ( 2 )\end{matrix}$$

where μ, q and k are constants, which may be empirically determined, and τₓ is the intrinsic period of oscillation of the circadian cycle (in hours). The driving input B is related to light intensity I as will be described further below. In one particular embodiment, μ=0.13, q=1/3, k=0.55 and τₓ=24.2 hours.

The parameter B is the driving input for the Jewett-Kronauer model equations (1) and (2). Accordingly, given any value of the driving input B and historical information about the circadian state variables (x, x_{c}) (i.e. initial conditions), equations (1) and (2) may be used to predict future values of the circadian state variables (x, x_{c}). As can be seen by examining equations (1) and (2), the circadian state variables (x, x_{c}) comprise oscillating functions which may be approximately sinusoidal in nature and which are approximately 90° (or ¼ period) out of phase with each another.

The Jewett-Kronauer model also comprises another state variable n which is not specifically related to the individual's circadian state, but rather is related to the individual's dynamic response to light. In accordance with the Jewett-Kronauer model, the state variable n is related to a parameter a which is an indication of an individual's perception of a given light intensity I. It will be appreciated that the human eye and brain have a nonlinear response to light intensity. For example, the introduction of a single candle into an otherwise dark room causes a dramatic increase in an individual's ability to perceive his or her environment, whereas the addition of one extra flourescent tube into a room already lit with five such tubes makes comparatively little difference. The response α of the human eye to light with an intensity I may be modeled by:

$$α = {{α}_{0} ( \frac{I}{9500} )}^{p} \qquad ( 3 )$$

where I is measured in lux and αₒ and p are constants which may be empirically determined. In one particular embodiment, αₒ=0.16 and p=0.6.

The third state variable n of the Jewett-Kronauer model may then be expressed in terms of α, the driving input B and the circadian state variables (x, x_{c}):
\dot{n}=60[α(1−n)−βn] (4)
B=Gα(1−n)(1−mx)(1−mx_{c}) (5)
where β and G are constants which may be empirically determined. In one particular embodiment, β=0.013 and G=19.875.

The driving input B is related to light intensity I. It is useful for the purpose of employing the Jewett-Kronauer model in real world applications to provide transformations back and forth between the domain of driving input B and the domain of light intensity I. Such transformations allow systems which make use of the Jewett-Kronauer model to work in either the driving input domain B or in the light intensity domain I. Transformations back and forth between the driving input domain B and the light intensity domain I may involve one or more simplifying approximations.

Some embodiments of the invention make use of a particular transformation between the driving input domain B of the Jewett-Kronauer model and the light intensity domain I, the derivation of which is presented below. Equation (5) may be rearranged as:

$$α = \frac{B}{G ( 1 - n ) ( 1 - mx ) ( 1 - {mx}_{c} )} \qquad ( 6 )$$

As can be appreciated by analyzing equation (4), the state variable n has a nonlinear dependence on the value of α. In some embodiments of the present invention, light pulses are applied to individuals to controllably alter their circadian cycles. In particular applications of the invention, light pulses of interest typically have durations greater than two hours. In these circumstances, it can assumed that the decaying exponential dependence of n on α (as described by equation (4)) will reach a steady state within a period of time significantly less than the duration of the light pulses. Accordingly, in such applications, the time derivative of n can be approximated as zero, such that equation (4) may be solved for an approximate value of n:

$${n}_{approx} = {lim}_{t -> ∞} n = \frac{α}{α + β} \qquad ( 7 )$$

Substituting this approximate value of n from equation (7) into equation (6) yields:

$$\begin{matrix}α \approx \frac{B}{G ( 1 - \frac{α}{α + β} ) ( 1 - mx ) ( 1 - {mx}_{c} )} \\ = \frac{β B}{G β ( 1 - mx ) ( 1 - {mx}_{c} ) - B}\end{matrix} \qquad ( 8 )$$

In addition, equation (3) may be rearranged as:

$$I = 9500 {( \frac{α}{{α}_{0}} )}^{\frac{1}{p}} \qquad ( 9 )$$

and substituting equation (8) into equation (9) yields:

$$I \approx 9500 {( \frac{β B}{{α}_{0} [ G β ( 1 - mx ) ( 1 - {mx}_{c} ) - B ]} )}^{\frac{1}{p}} \qquad ( 10 )$$

Equation (10) represents a transformation function of the form I=f(x, x_{c}, B) which can be used in conjunction with the circadian state variables (x, x_{c}) to transform a value in the driving input domain B into a corresponding value in the light intensity domain I.

Using the same approximation for n from equation (7) and substituting it into equation (5) yields:

$$B \approx G \frac{α β}{α + β} ( 1 - mx ) ( 1 - {mx}_{c} ) \qquad ( 10 A )$$

Then, substituting equation (3) into equation (10A) yields:

$$B = \frac{G {{α}_{o} ( \frac{I}{9500} )}^{p}}{{{α}_{o} ( \frac{I}{9500} )}^{p} + β} ( 1 - mx ) ( 1 - {mx}_{c} ) \qquad ( 10 B )$$

Equation (10B) represents a transformation function of the form B=g(x, x_{c}, I) which can be used in conjunction with the circadian state variables (x, x_{c}) to transform a value in the light intensity domain I into a corresponding value in the driving input domain B.

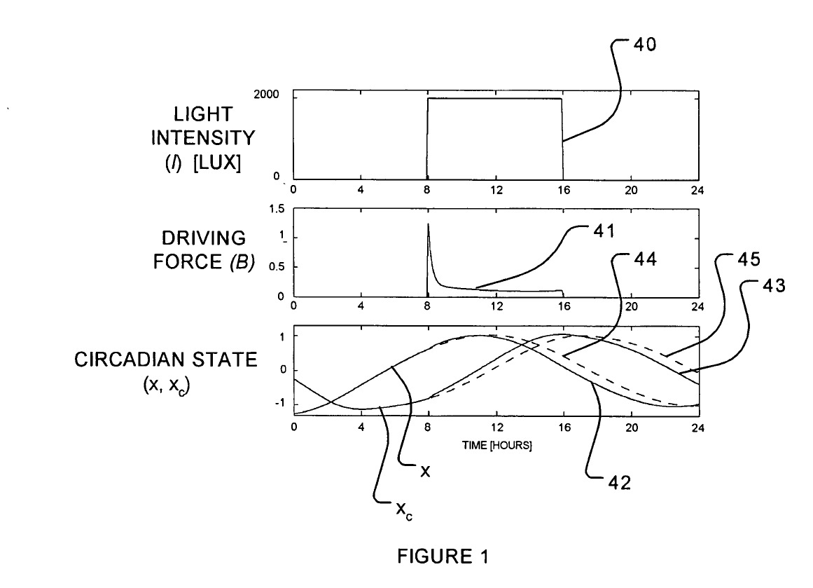

FIG. 1 depicts the response of the circadian state variables (x, x_{c}) defined by the Jewett-Kronauer model equations (1)-(10B) to a pulse of light 40. More particularly, FIG. 1 depicts curves 44, 45, which respectively represent the nominal circadian state variables (x, x_{c}) predicted by model equations (1) and (2) under zero light conditions, and curves 42, 43, which respectively represent the circadian state variables (x, x_{c}) predicted by model equations (1) and (2) in response to light pulse 40. As can be seen by comparing curves 42, 43, 44, 45, light pulse 40 causes a phase shift in circadian state variables (x, x_{c}).

The direction of the phase shift caused by light pulse 40 on circadian state variables (x, x_{c}) depends on the actual phase of these circadian state variables (x, x_{c}) in relation to the timing of light pulse 40. In the example graphs of FIG. 1, after the application of light pulse 40 at t=8 hours, curve 43 leads curve 45, which indicates that light pulse 40 has caused an advance in the phase of circadian state variable x_{c}. FIG. 1 also depicts a signal 41 representative of the driving input B associated with light pulse 40. As shown by curve 41 of FIG. 1, driving input B exhibits a relatively sharp peak at the outset of light pulse 40 (i.e. around t=8 hours) and then decays over time to a steady state value, until light pulse 40 is terminated, at which point driving signal B returns to zero.

The Jewett-Kronauer model represented by equations (1)-(10B) is a nonlinear model. In particular, equations (1) and (2), which describe the circadian state variables (x, x_{c}), comprise a pair of nonlinear equations expressed in terms of the driving variable B and the circadian state variables (x, x_{c}). Some embodiments of the present invention make use of linearizing transformations or manipulations which allow a nonlinear model to be approximated by a linear model. Such manipulations may involve nonlinear state feedback compensation and/or various linearizing approximations.

Particular embodiments of the invention employ linearizing approximations to provide linearized versions of equations (1) and (2) of the Jewett-Kronauer model. With respect to equation (1), it may be observed that there are two higher order (i.e. nonlinear) terms of the circadian state variable x, both of which are scaled by the factor μ. It may also be observed that equation (1) is an odd function of x, which has an integral of zero over the range of x (xε[−1,1]). Because these higher order terms introduce relatively small contributions that average to zero over the range of x, one possible approximation for equation (1) involves setting μ=0. With regard to equation (2), there are two higher order terms which are functions of Bx_{c} and Bx and which are scaled by scaling factors q and k respectively. As with the approximation for equation (1), one possible approximation for equation (2) involves setting the scaling factors q and k to zero. These approximations to equations (1) and (2) (i.e. setting scaling factors μ, q and k equal to zero) result in the following linearized state space equations:

$$\begin{matrix}[ \begin{matrix}\dot{x} \\ \dot{{x}_{c}}\end{matrix} ] = [ \begin{matrix}0 & \frac{π}{12} \\ {( \frac{24}{{τ}_{x} ( 0.99729 )} )}^{2} & 0\end{matrix} ] [ \begin{matrix}x \\ {x}_{c}\end{matrix} ] + [ \begin{matrix}\frac{π}{12} \\ 0\end{matrix} ] B & ( 11 ) \\ [ {x}_{out} ] = [ \begin{matrix}1 & 0\end{matrix} ] [ \begin{matrix}x \\ {x}_{c}\end{matrix} ] & ( 12 )\end{matrix}$$

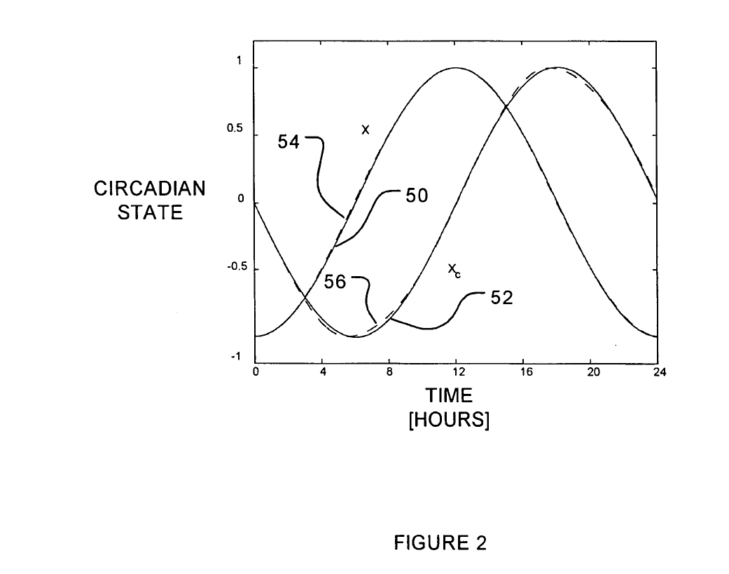

FIG. 2 depicts a comparison of nonlinear model equations (1) and (2) and linearized model equations (11) under conditions where B=0 (i.e. no driving input). Solid curves 50, 52 respectively represent the circadian state variables (x, x_{c}) calculated using equations (1) and (2) and dashed curves 54, 56 respectively represent the circadian state variables (x, x_{c}) calculated using linearized approximation equations (11). It can be seen from curves 50, 52, 54, 56, that the linearized approximation equations (11) produce results which are fairly close to those of model equations (1) and (2), at least for the condition where B=0.

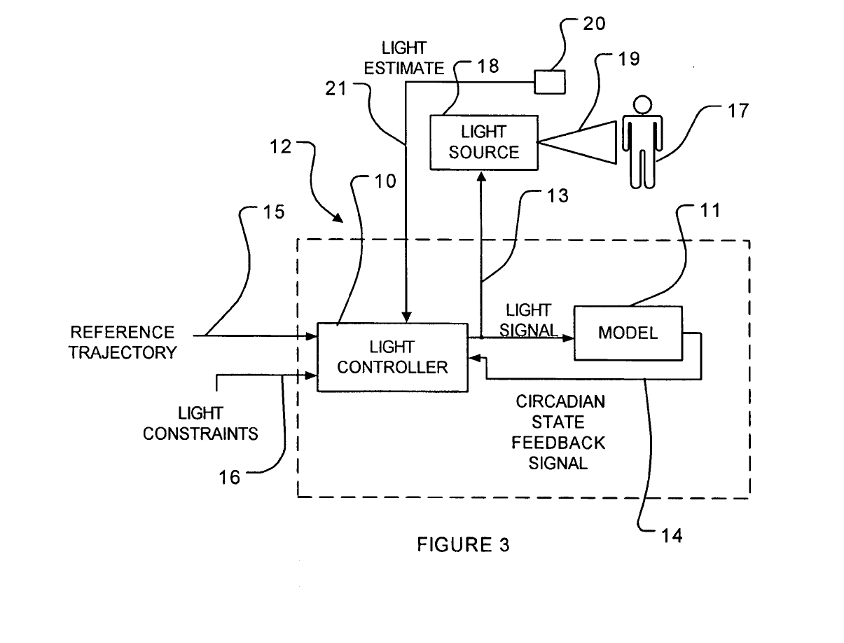

FIG. 3 is a block diagram of a system 12 for controllably adjusting a circadian cycle of a subject 17 in accordance with a particular embodiment of the invention. System 12 comprises a controller 10 which receives reference trajectory input 15, light constraint inputs 16, light estimate input 21 and circadian state feedback signal 14. In response to these inputs, controller 10 outputs a light control signal 13. Light control signal 13 may control various characteristics of light 19 output by one or more light sources 18. In some embodiments, light control signal 13 is representative of the intensity I of light 19 output by light source(s) 18. The light intensity I may be measured in lux. When light 19 is applied to subject 17, the circadian pacemaker cycle of subject 17 is altered.

In the FIG. 3 embodiment, system 12 comprises a circadian state feedback signal 14 that is generated by model 11. Model 11 preferably comprises a mathematical model of the response of a human circadian state to a given stimulus. In the FIG. 1 embodiment, model 11 receives input information from light control signal 13 which represents the intensity I of light stimulus 19. Using light control signal 13 and historical circadian state information (which may be stored in a memory (not shown)), model 11 predicts the current circadian state of subject 17 in response to light stimulus 19 and feeds this information back to controller 10 as circadian state feedback signal 14.

In one particular embodiment, model 11 comprises the Jewett-Kronauer model described above in equations (10)-(10B). In another particular embodiment, model 11 comprises the Jewett-Kronauer model described above in equations (1)-(10B), except that linearized equation (11) replaces nonlinear equations (1) and (2). In such embodiments, model 11 predicts one or more of the circadian state variables (x, x_{c}) and feeds this information back to controller 10 as circadian state feedback signal 14. Those skilled in the art will appreciate that model 11 may make use of transformations between the intensity domain I of light control signal 13 and the driving input domain B of the Jewett-Kronauer model.

Reference trajectory input 15 is preferably representative of a target time varying circadian pacemaker cycle. System 12 controllably adjusts the circadian pacemaker cycle of subject 17 to track reference trajectory input 15. In some embodiments, reference trajectory input 15 comprises desired values of one or more of the circadian state variables (x, x_{c}) of the Jewett-Kronauer model described above. Light constraint inputs 16 may comprise minimum and/or maximum light intensity levels which may be applied to subject 17. Such minimum and maximum levels may be related to actual physical constraints of light source(s) 18 or to other levels, such as levels designed to promote the comfort of subject 17 for example. Light estimate input 21 is an optional input which comprises an estimate of light 19 experienced by subject 17. Light estimate input 21 may be in the intensity domain I and may be measured in lux. Light estimate input 21 comes from light estimator 20. In some embodiments, light estimator 20 comprises one or more sensors for measuring light 19. In other embodiments, light estimator 20 comprises a model which generates a signal representative of the light experienced by subject 17 and feeds this signal back to controller 10 as light estimate input 21. Such a model may take into account the current light signal 13, other sources of light (not shown), such as sunlight, ambient light, background artificial light, and other factors, such as time of day, date, location, activity patterns, and amount of light that will be incident on the retinas of subject 17.

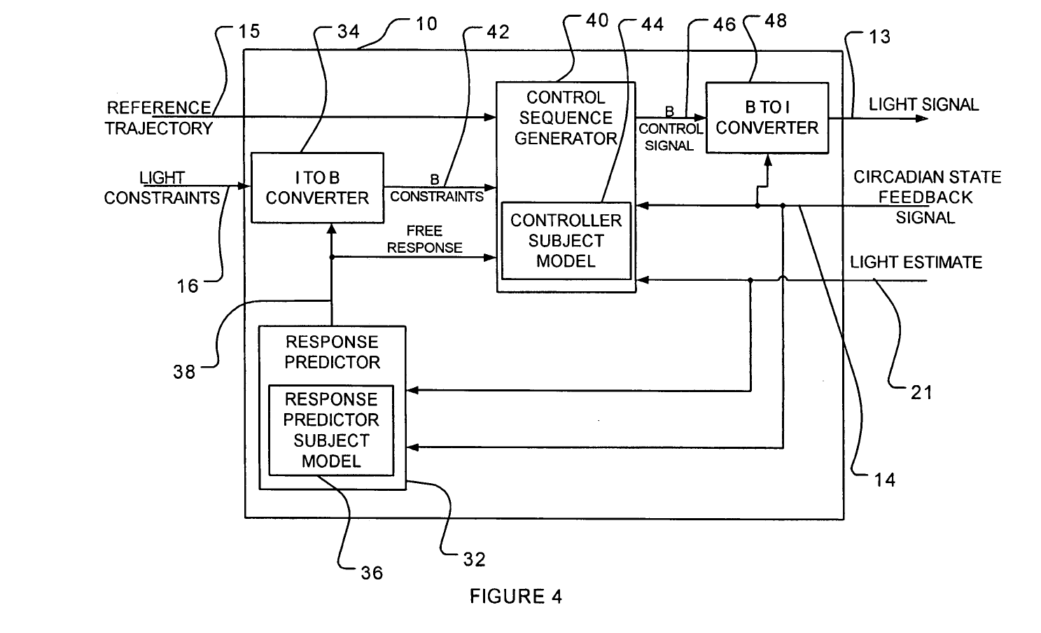

FIG. 4 depicts a particular embodiment of controller 10 of system 12 in more detail. Controller 10 may be implemented using various types of programmable controllers or processors. For example, controller 10 may comprise a programmable computer, an embedded processor or the like. Controller 10 may comprise more than one such processor. Controller 10 may also include memory (not shown) which stores program information and the like. Controller 10 receives: reference trajectory input 15, light constraint signals 16, circadian state feedback signal 14 and light estimate input 21. In response to these inputs, controller 10 generates light control signal 13 which (when applied to light source(s) 18) causes the circadian pacemaker cycle of subject 17 to track reference trajectory 15.

In the FIG. 4 embodiment, controller 10 comprises an I to B converter 34 and a B to I converter 48. B to I converter 48 transforms drive control signal 46 in the drive signal input domain B into a corresponding light control signal 13 in the intensity domain I. B to I converter 48 may make use of the transformation expressed in equation (10). To perform its transformation, B to I converter 48 has access to circadian state feedback signal 14 which may comprise one or more of the circadian state variables (x, x_{c}). I to B converter 34 transforms light constraint inputs 16 in the intensity domain I into corresponding drive signal constraint inputs 42 in the drive signal input domain B. I to B converter 34 may make use of a transformation of the form expressed in equation (10B). As shown in FIG. 4, I to B converter 34 has access to free response information 38. As will be explained further below, free response information 38 may comprise information representative of free response values for the Jewett-Kronauer state space variables (x, x_{c}).

Control system 10 comprises response predictor 32 which comprises a response predictor subject model 36. Response predictor 32 receives light estimate input 21 and circadian state feedback signal 14 and generates free response information 38. In preferred embodiments, free response information 38 comprises a prediction of the future circadian state of individual 17 under the assumptions that the initial circadian state of individual 17 is indicated by circadian state feedback signal 14 and the current light level I indicated by light estimate input 21 remains constant. Response predictor 32 calculates the free response future circadian state of individual 17 (i.e. free response information 38) out to a control horizon H.

In particular embodiments, response predictor 32 calculates a plurality of future free response values of the circadian state of individual 17 after each of a number p of discrete sampling intervals between the current time and the control horizon H. In general, the control horizon H and the sampling interval may be of any duration depending on the application, processor resources, etc. In some preferred embodiments, the sampling interval is in a range of 1 minute to 1 hour. In particularly preferred embodiments, each sampling interval is in a range of 10-30 minutes. In some preferred embodiments, the length of the control horizon H is in a range of 12-72 hours. In particularly preferred embodiments, the length of the control horizon H is in a range of 24-36 hours.

In the FIG. 4 embodiment, response predictor 32 comprises a response predictor subject model 36 which may be used to generate free response information 38. In one particular embodiment, response predictor subject model 36 comprises the Jewett-Kronauer model described above in equations (1)-(10B). In another particular embodiment, response predictor subject model 36 comprises the Jewett-Kronauer model described above in equations (1)-(10B), except that linearized equation (11) replaces nonlinear equations (1) and (2). In such embodiments, response predictor subject model 36 uses light estimate input 21 and circadian feedback signal 14 to predict the free response of one or more of the circadian state variables (x, x_{c}) out to the control horizon H under the assumptions described above. In some embodiments, free response information 38 takes the form of a matrix comprising free response values of one or more of the circadian state variables (x, x_{c}) at p discrete sampling intervals. Those skilled in the art will appreciate that response predictor subject model 36 may make use of transformations between the intensity domain I of light estimate input 21 and the driving input domain B of the Jewett-Kronauer model.

Controller 10 also comprises a control sequence generator 40 which comprises a controller subject model 44. In one particular embodiment, controller subject model 44 comprises the Jewett-Kronauer model described above in equations (1)-(10B). In a preferred embodiment, controller subject model 44 comprises the Jewett-Kronauer model described above in equations (1)-(10B), except that linearized equation (11) replaces nonlinear equations (1) and (2).

Control sequence generator 40 receives B domain constraints 42 (i.e. light constraints 16 converted from the intensity domain I to the B domain by I to B converter 34), free response information 38, light estimate input 21 and circadian state feedback signal 14 and uses these inputs to generate a series of control moves in the B domain (not shown). In preferred embodiments, control sequence generator 40 determines a series of “optimal” control moves in the B domain (not shown) which extend over a time period beginning at the current time and extending out to the control horizon H. As explained further below, this series of control moves is “optimized” by minimizing a cost function over the time period between the current time and the control horizon H. At each sampling interval, control sequence generator 40 outputs the first control move of this series as B domain control signal 46. B domain control signal 46 is converted by B to I converter 48 into light control signal 13 in the intensity domain I. As discussed above, light control signal 13 controls the operation of light source(s) 18 and the amount of light 19 experienced by subject 17.

In some embodiments, the cost function used by control sequence generator 40 is a least squares cost function. In one particular embodiment, the cost function used by control sequence generator 40 is expressed as:

$$J = {\sum }_{k = 1}^{p} {‖ δ ( k ) [ \hat{x} ( t + k \vert t ) - r ( t + k ) ] ‖}^{2} + {\sum }_{k = 1}^{p} {‖ λ ( k ) B ( t + k - 1 ) ‖}^{2} \qquad ( 13 )$$

where p is the number of samples between the current time t and the control horizon H, δ(k) is a weighting factor associated with the tracking error (i.e. the difference between the circadian state \hat{x} predicted by controller subject model 44 and the reference circadian state r described by reference trajectory 15), λ(k) is a weighting factor associated with the cost of the driving input B and k is an index variable that indexes a particular sampling interval within the control horizon H. In some embodiments, weighting factors δ(k) and λ(k) are constant. In other embodiments, however, weighting factors δ(k) and λ(k) are functions of k, such that additional weight may be applied to tracking particular points on the reference trajectory 15, such as the maxima and minima of reference trajectory 15 or the zero crossing points of reference trajectory 15 for example.

Control sequence generator 40 may make use of controller subject model 44 and the inputs described above to determine the “optimal” control moves (in the B domain) which will minimize the cost function of equation (13) over the control horizon H. Such control moves may comprise a series of B values (i.e. at each sampling time) from the current time and extended out to the control horizon H. After each sample interval, the control horizon H is extended by the time of one sampling interval and the control sequence generator 40 recalculates the “optimal” moves.

In practical situations, there will be light constraints 16 on the light intensity I. Such light constraints 16 may comprise minimum and maximum light intensity levels which may be related to actual physical constraints of light source(s) 18 or to other levels, such as levels designed to promote the comfort of subject 17 or the sleep/wake schedule of subject 17 for example. Light constraints 16 in the intensity domain I are converted to B domain constraints 42 by I to B converter 34 and are provided to control sequence generator 40. Preferably, light constraints 16 (and corresponding B domain constraints 42) comprise light constraint values for times extending out to the control horizon H. In some embodiments, light constraints 16 (and corresponding B domain constraints 42) may be constant.

As discussed above, I to B converter 34 may make use of a transformation of the form of equation (10B) to transform light constraints 16 in the intensity domain into corresponding B domain constraints 42. However, calculation of future B domain constraints 42 from future I domain constraints 16 in accordance with equation (10B) requires information about the future values of circadian state variables (x, x_{c}). This creates an iterative problem, because future values of the circadian state variables (x, x_{c}) depend in turn on the future B domain constraints 42.

In the FIG. 4 embodiment, I to B converter 34 overcomes this iterative problem by making use of the free response information 38 as an approximation of the future values of circadian state variables (x, x_{c}). As discussed above, the free response information 38 preferably comprises a prediction of the future circadian state of individual 17 under the assumptions that the initial circadian state of individual 17 is indicated by circadian state feedback signal 14 and the current light intensity I indicated by light estimate input 21 will remain constant. In some embodiments, free response information 38 takes the form of a matrix comprising free response values of one or more of the circadian state variables (x, x_{c}) at p discrete sampling intervals.

As discussed above, intensity domain light constraints 16 preferably comprise maximum and minimum light intensity levels. These maximum and minimum light intensity levels correspond with maximum and minimum B domain levels, Bₘₐₓ and Bₘᵢₙ. Once the current and future B domain constraints 42 are determined by I to B converter 34, control sequence generator 40 determines the “optimal” control moves by minimizing a cost function in subject to the constraints:
Bₘᵢₙ(k)≦B(k)≦Bₘₐₓ(k) for k=1 . . . p (14)
In embodiments where the cost function is given by equation (13), control sequence generator 40 selects B values for each sampling interval between the current time and extending out to the control horizon H (i.e. where k=p) which minimize the cost function of equation (13) within the constraints given by equation (14). Those skilled in the art of control systems and model-based predictive control are familiar with various techniques and algorithms for numerically or otherwise solving this minimization problem. Typically, such techniques involve constrained quadratic programming methods.

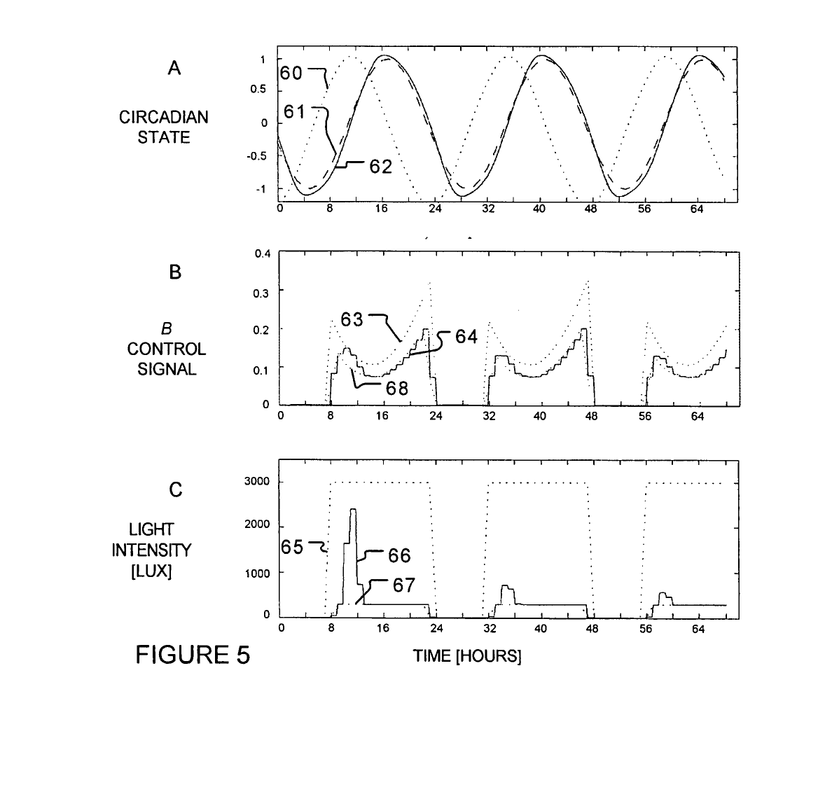

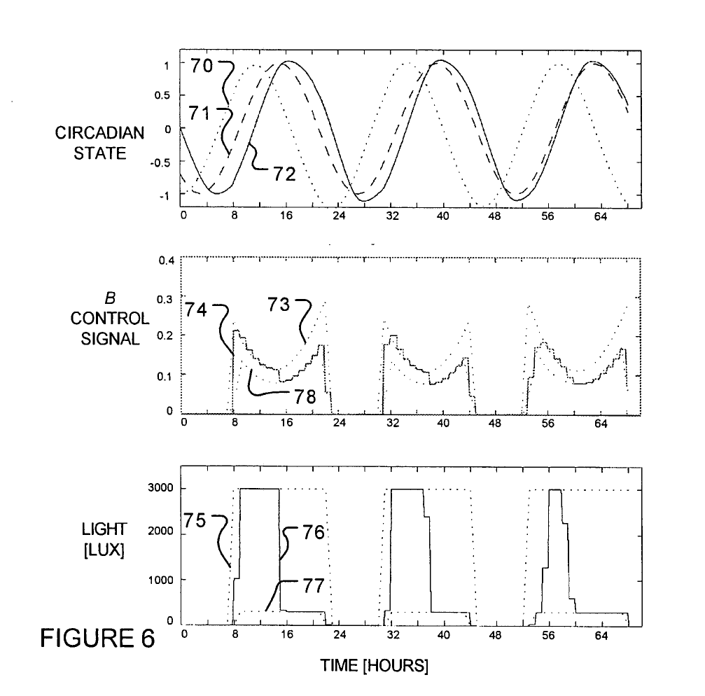

FIGS. 5 and 6 depict a number of examples of the operation of system 12 to controllably adjust the circadian pacemaker cycle of subject 17 to track a reference trajectory 15 in the presence of lighting constraints 16. In the examples of FIGS. 5 and 6, it is assumed that subject 17 is an astronaut in orbit. Astronauts in orbit may experience cycles of sunlight and darkness with greatly reduced periods. It may be desirable to artificially maintain an approximately 24 hour circadian pacemaker cycle to ensure optimal alertness and increased quality of sleep during missions.

For the purposes of the examples illustrated in FIGS. 5 and 6, it is assumed that the intensity of light 19 (FIG. 1) is constrained in the following manner:
(i) the range of light extends from darkness (I=0 lux) to a maximum light intensity (I=10,000 lux);
(ii) subject 17 sleeps between time t=0 and time t=8 hours (and every 24 hours thereafter) and no light can be applied during sleep (I=0 lux);
(iii) a minimum amount of light (I≧300 lux) is necessary to perform daily activities while awake (i.e. not sleeping); and
(iv) the minimum amount of light is reduced (I≧100 lux) for the first hour after rising and the last hour before sleeping.
   These constraints are represented in FIG. 5 by the dashed curves 65 and 67, where curve 65 represents the maximum light intensity Iₘₐₓ and curve 67 represents the minimum light intensity Iₘᵢₙ. It is also assumed that the sunlight experienced by the astronaut is insignificant in comparison to applied light 19.

In the example depicted in FIG. 5, subject 17 is maintaining a regular 24 hour schedule of 8 hours asleep and 16 hours awake and the subject's circadian pacemaker cycle(represented by circadian state variable x and curve 62) is initially closely synchronized to the desired reference trajectory 61. Curve 60 represents the circadian state variable x_{c} as a function of time. In the example of FIG. 5, circadian state variable x_{c} is not controlled. During operation, I to B converter 34 converts the intensity domain light constraints Iₘₐₓ (curve 65) and Iₘᵢₙ (curve 67) into B domain constraints Bₘₐₓ (curve 63) and Bₘᵢₙ (curve 68). As discussed above, control sequence generator 40 generates an optimal set of control moves in the B domain which will allow circadian state variable x (curve 62) to track reference trajectory 61 by minimizing a cost function subject to the B domain constraints. B domain control signal 46 is represented in FIG. 5 by curve 64. As discussed above, B to I converter 48 converts B domain control signal 46 (curve 64) into light control signal 13 (represented in FIG. 5 by curve 66). As expected from the regular sleep pattern of subject 17, the subject's circadian pacemaker cycle (as represented by circadian state variable x and curve 62) continues naturally on its 24 hour rhythm, and little additional light 66 is required to maintain it.

In the example illustrated in FIG. 6, subject 17 receives a mission requirement that involves shifting the waking hours ahead by 3 hours. The shift must occur within 2 days. Accordingly, it is desirable to shift the circadian pacemaker cycle of subject 17 such that within two days, the subject's circadian pacemaker cycle (as represented by state variable x and curve 72) tracks the reference trajectory 71. It can be seen that initially (i.e. at time t=0), reference trajectory 71 has a three hour phase lead with respect to the subject's circadian pacemaker cycle as represented by circadian state variable x (curve 72).

Curve 70 represents the circadian state variable x_{c} as a function of time. In the example of FIG. 6, circadian state variable x_{c} is not controlled. The light constraints in the FIG. 6 example are substantially similar to those in the example of FIG. 5, with Iₘₐₓ represented by curve 75, Iₘᵢₙ represented by curve 77, Bₘₐₓ represented by curve 73 and Bₘᵢₙ represented by curve 78. Control sequence generator 40 determines the optimum control moves in the B domain which will controllably adjust circadian state variable x (curve 72) towards reference trajectory 71 by minimizing a cost function subject to the B domain constraints. B domain control signal 46 is represented in FIG. 6 by curve 74. As discussed above, B to I converter 48 converts B domain control signal 46 (curve 74) into light control signal 13 (represented in FIG. 6 by curve 76). As shown in FIG. 6, by the third day, light control signal 13 (curve 76) causes an application of light 19 to subject 17 which controllably adjusts the subject's circadian pacemaker cycle (as represented by circadian state variable x and curve 72) to successfully track reference trajectory 71 by the third day. Comparing curve 76 of FIG. 6 with curve 66 of FIG. 5, it can be seen that considerably more light is required to substantially shift a subject's circadian pacemaker cycle.

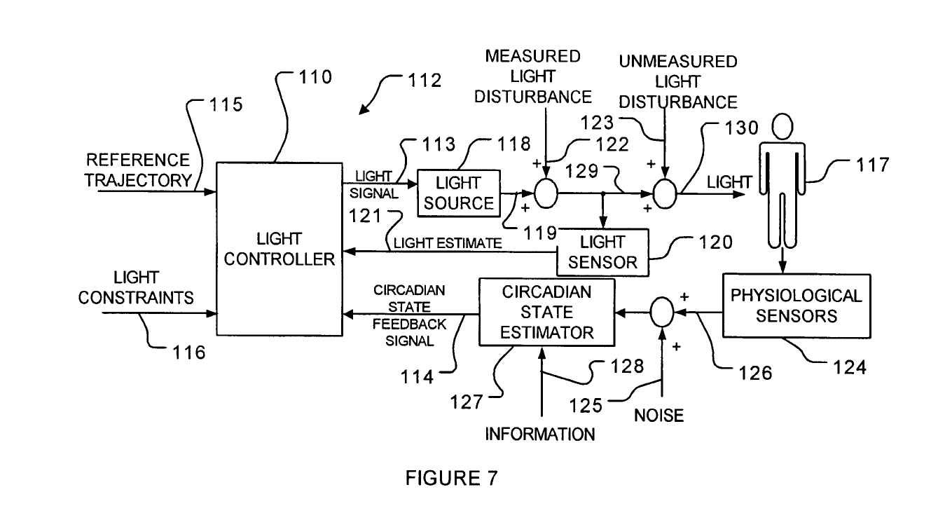

FIG. 7 depicts a system 112 according to another embodiment of the invention. System 112 is similar in many respects to system 12 of FIGS. 3 and 4. Features and components of system 112 that are similar to features and components of system 12 are given similar reference numerals preceded by a “1”. System 112 differs from system 12 in that system 112 comprises one or more physiological sensors 124 and a circadian state estimator 127 in the place of model 11 (FIG. 3). Together, physiological sensors 124 and circadian state estimator 127 obtain a circadian state feedback signal 114 that is based, at least in part, on the physiological parameters sensed by sensors 124.

Physiological sensors 124 sense one or more physiological parameters from subject 117. Preferably, the physiological parameters sensed by sensors 124 are correlated to or otherwise related to the circadian state of subject 117. Preferably, the physiological parameters sensed by sensors 124 vary periodically in a manner that is related to the periodic variation of the circadian cycle of subject 17. Examples of physiological parameters include, without limitation: heart rate, core body temperature, respiration, endocrine function levels, physical activity levels, blood pressure, blood oxygen concentration and/or skin temperature.

The sensed values 126 of the physiological sensors 124 are provided to circadian state estimator 127. In some embodiments, the sensed values 126 of the physiological sensors 124 are communicated to circadian state estimator 127 via a wireless communication means (not shown). This allows subject 117 to be mobile. In other embodiments, the sensed values 126 of the physiological sensors 124 are communicated to circadian state estimator 127 via one or more corresponding wires. Noise 125 may appear parasitically in the sensed values 126 of the physiological sensors 124 prior to their reaching circadian state estimator 127. Noise 125 may be modeled as part of circadian state estimator 127 and/or in the model(s) associated with light controller 110. In some embodiments, noise 125 is modeled stochastically.

Circadian state estimator 127 receives the sensed values 126 of the physiological sensors 124 and uses this information to determine an estimate of the circadian pacemaker state of subject 117. This estimate of the circadian pacemaker state of subject 117 is provided to controller 110 as circadian state feedback signal 114. Preferably, circadian state feedback signal 114 generated by circadian state estimator 127 comprises one or more of the circadian state variables (x, x_{c}) of the Jewett-Kronauer model. In some embodiments, circadian state estimator 127 comprises a model (not shown) which relates the values of one or more physiological parameters to the circadian state of subject 117. Such a model may be empirically determined and may use the sensed values 126 from physiological sensors 124.

Circadian state estimator 127 may receive other information 128 which it uses to help determine an estimate of the circadian state of subject 117. Information 128 may include one or more parameters that may be entered by subject 117, such as the sleep/wake schedule of subject 117 for example. Information 128 may include one or more parameters that are particular to subject 117, such as an indicator of the responsiveness of subject 117 to light (or other stimulus) for example. Information 128 may include one or more parameters associated with the environment in which subject 117 spends his or her time, such as temperature, time of day, time zone and background stimulus level for example.

As with system 12 of FIGS. 3 and 4, system 112 comprises one or more light sources 118 that output light 119 in response to light control signal 113 from controller 110. In the illustrated embodiment, system 112 comprises one or more light sensors 120 which measure light intensity 129 present in the environment of subject 117. Light intensity 129 measured by light sensor(s) 120 represents an estimate of the light intensity experienced by subject 117. Light 129 differs from the light output 119 from light source(s) 118, as measured light disturbances 122 (for example, from other light sources) may be measured as a part of light 129. In addition, light 129 differs from the light 130 actually experienced by subject 117, as unmeasured light disturbances 123 may not be measured by light sensor 120. Light 129 measured by light sensor 120 is fed back to controller 110 as light estimate signal 121. Controller 110 uses light estimate signal 121 as discussed above in relation to controller 10. In some embodiments, controller 110 uses light estimate signal 121 to adjust light control signal 113 to compensate for the addition of light disturbances 122 and/or 123.

In other respects, system 112 is substantially similar to system 12 of FIGS. 3 and 4. In particular, the components and operation of light controller 110 are substantially similar to the components and operation of light controller 10 shown in FIG. 4 and described in detail above.

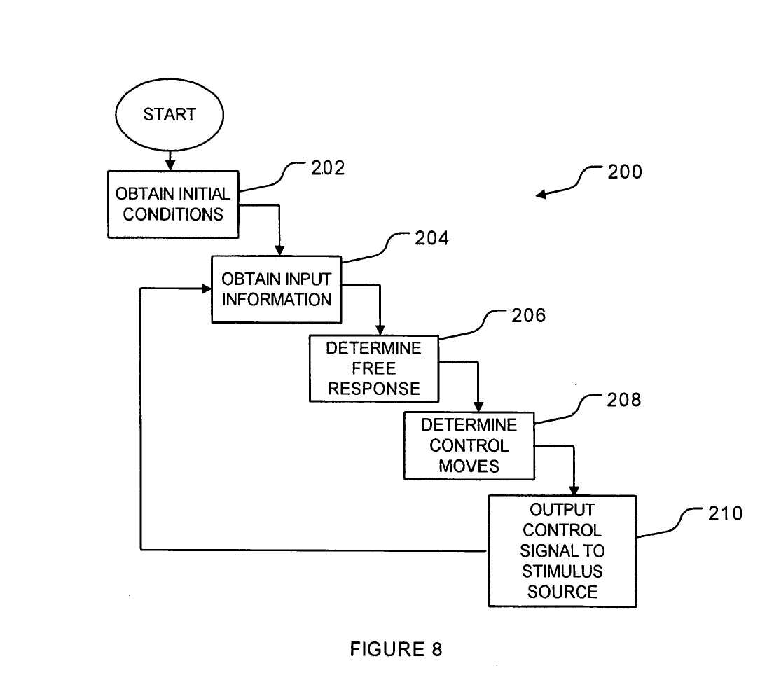

FIG. 8 schematically depicts a method 200 for controlling a circadian cycle of a subject in accordance with a particular embodiment of the invention. Method 200 may be executed by controller 10, 110 of systems 12, 112. After commencing, method 200 proceeds to block 202, which involves determining the initial conditions. The initial conditions may include the initial circadian state of the subject. In one particular embodiment, the initial conditions comprise values of one or more of the state variables (x, x_{c}, n) of the Jewett-Kronauer model. In other embodiments, the initial conditions determined in block 202 comprise values of state variables associated with other models. The initial conditions determined in block 202 may be used by controller 10, 110 of systems 12, 112. In particular, the initial conditions may be used by response predictor 32 as the initial conditions for response predictor subject model 36 and/or by control sequence generator 40 as the initial conditions for controller subject model 44. The initial conditions determined in block 202 may also be used as the initial conditions for model 11 of system 12.

In some embodiments, determining the initial conditions in block 202 involves arbitrarily assigning initial conditions. In other embodiments, determining the initial conditions in block 202 involves estimating the circadian state of the subject based on measurement of one or more physiological parameters. Such estimation may be substantially similar to the estimation performed by circadian state estimator 127 described above and may incorporate other information similar to information 128 described above. In still other embodiments, determining the initial conditions in block 202 may comprise determining an initial time and using a population average circadian state for the initial time.

Method 200 then proceeds to block 204 where input information is obtained. As shown best in FIG. 4, the input information includes the desired reference trajectory 15, light constraints 16, light estimate 21 and circadian state feedback signal 14. In particular embodiments which employ the Jewett-Kronauer model, reference trajectory 15 and circadian state feedback signal 14 may comprise one or more of the Jewett-Kronauer circadian state variables (x, x_{c}). In other embodiments, reference trajectory 15 and circadian state feedback signal 14 comprise values of state variables associated with other models. In some embodiments (such as system 12 of FIG. 3), circadian state feedback signal 14 is derived from a model 11. In other embodiments, (such as system 112 of FIG. 7), circadian state feedback signal 14 is derived from sensed physiological parameters 126 and a corresponding estimate of the subject's circadian state. Light constraints 16 may comprise minimum and maximum light levels. In some embodiments, minimum light levels are determined with reference to varying expected ambient light levels. As discussed above, light estimate 21 may comprise a sensed value of the light experienced by the subject and/or an estimated value of the light experienced by the subject.

Block 206 of method 200 involves determining the free response of the subject out to a control horizon H. As discussed above in the description of FIG. 4, determining the free response in block 206 may be performed by response predictor 32. Response predictor 32 may use a response predictor subject model 36. Determining the free response in block 206 may involve the assumptions that the current circadian state of the subject (as determined by circadian state feedback signal 14) represents the initial conditions and that current light control signal 13 remains constant.

In block 208, method 200 involves determining the optimal control moves out to the control horizon H. As discussed above in the description of FIG. 4, determining the optimal control moves in block 208 may be performed by control sequence generator 40. Control sequence generator 40 may make use of a controller subject model 44, which may be linearized. Determining the optimal control moves in block 208 may comprise minimizing a cost function which has a term associated with tracking error and a term associated with the amount of control signal required. The cost function may comprise weighting factors which may be constant or time dependent. The cost function incorporates each control move (i.e. one for each sampling interval) as independent variables. Minimization of the cost function is accomplished by finding the set of control moves that produces a minimum. This minimization operation may be performed by expressing the equations for the cost function and the constraints as a linear matrix inequality and then solving the linear matrix inequality in accordance with one of many well known numerical techniques.

Model based predictive control techniques of the type employed in blocks 206 and 208 of method 200 are well known to those skilled in the art and are described comprehensively in “Predictive Control with Constraints”, Jan Maciejowski, 2002 (Pearson Education POD), which is hereby incorporated herein by reference.

Once the optimal control moves are determined in block 208, method 200 proceeds to block 210 which involves outputting a control stimulus signal. As discussed above, the control stimulus signal may comprise a light control signal 13. Preferably, the control stimulus signal comprises the first control move of the optimal series of control moves determined in block 208.

After outputting a control stimulus signal in block 210, method 200 loops back to block 204 and then repeats itself. Preferably, the loop which comprises blocks 204, 206, 208 and 210 is performed at least once every sampling interval.

The systems and methods of the current invention have useful application in a variety of fields including, without limitation: controlling workplace lighting to maintain maximum employee performance; synchronizing multiple subjects to a common circadian cycle to improve operational efficiency; modifying circadian cycles of individuals undergoing medical therapy to improve their response to pharmaceutical drugs; modifying circadian cycles of athletes to allow peak performance at certain times; controlling lighting in the home to help make the daily processes of waking and falling asleep more comfortable, and providing invidualized light recommendations or control for personal advisory systems.

Controlling workplace lighting by providing additional light or restricting the level of light as appropriate can create benefits for various types of employees. Example applications include workers in windowless or underground rooms, submarine personnel and miners who receive no natural light; hospital employees, utility employees and other types of shift workers who may receive some natural light at times that may be inappropriate to the workers' circadian pacemaker cycle; and workers (or other travelers) aboard aircraft or spacecraft, who experience abnormal natural light levels due to the speed of their travel in relation to the sun. The systems and methods of the invention could be applied to control the levels of light stimulus to help advantageously regulate the circadian cycles of these types of subjects.

Another application of the systems and methods of the invention comprises synchronization of the circadian cycles of multiple subjects. Scientific research often requires the performance of experiments such as controlled tests of a new technology, drug, method or product on a given sample group of animals. The sample group may consist of rats, mice, primates and other animals, or human subjects. In many instances, the response of each test subject in the sample group will vary depending on the state of the test subject's circadian cycle. It would be advantageous, therefore, to normalize the initial conditions of each test subject in a sample group by providing a means for synchronizing the circadian pacemaker cycle of each member of the sample group before conducting the experiment. The particular pattern of light exposure could be customized for each member of the sample group in order to most efficiently entrain their circadian pacemaker cycle to a desired common cycle.

In addition to improving the accuracy of scientific experimentation, synchronization of the circadian cycles of multiple human subjects may be beneficial in certain kinds of military operations. In particular, when a given military operation requires the simultaneous action of many individuals, the probability of success of the operation may be higher if the individual members of the force are all operating at maximum alertness and cognitive performance.

Another application of the systems and methods of the present invention relates to adjusting the circadian pacemaker cycle of individuals who compete in athletic events. In some circumstances, such athletes have to travel relatively long distances to compete and after traveling such a distance, it may be advantageous to control the circadian pacemaker cycle of such athletes to achieve maximum performance during the athletic event.

Another application of the systems and methods of the present invention is in the area of medical therapy. It is well understood by medical professionals that a patient undergoing pharmaceutical drug treatment responds differently to prescribed drugs at different states of her endogenous circadian pacemaker cycle. The methods and systems of the present invention could be used to control the patient's circadian pacemaker cycle such that the drugs could be administered at optimum times to have a desired effect.

Yet another area in which the systems and methods of the present invention may be useful is generally known as home automation. Basic lighting and sound systems used in homes require manual control to adjust the levels of intensity. Before during, and immediately after waking, and also in the evening immediately before and after falling asleep, such manual control may be inconvenient and uncomfortable. The systems and methods of the invention may be employed to control lighting and/or sound systems to turn them on or off at specific times or to increase/decrease their intensity at specific rates of change. Such systems could function as part of a waking alarm, generating a gradually increasing level of light (and/or other stimulus) at a specific time over a given interval of time, so as to beneficially modify the subject's circadian pacemaker cycle towards a cycle consistent with experiencing wakefulness at desired times.

Still another field of application of the systems and methods of the present invention relates to a personal ambient light advisory system, to assist with adjusting an individual's circadian pacemaker cycle. The invention could be applied as an off-line system that uses models to generate recommended light levels for a given set of constraints and objectives. The advisory system could optionally also incorporate real-time sensor input regarding light levels, activity patterns, or physiological parameters. The controller could then update its advice on light levels in real-time. Systems of these types could be implemented on personal digital assistants (PDA's), cellular telephones, personal computers or other portable devices with computational capability for example. Such an advisory system could comprise or otherwise be coupled with a light delivery apparatus such as a portable high-intensity light source, where it could provide recommendations for use or directly control the light output. Potential users of such systems include transmeridian airline travelers and shift work professionals.

Certain implementations of the invention comprise computer processors which execute software instructions which cause the processors to perform a method of the invention. The invention may also be provided in the form of a program product. The program product may comprise any medium which carries a set of computer-readable signals comprising instructions which, when executed by a data processor, cause the data processor to execute a method of the invention. The program product may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like or transmission-type media such as digital or analog communication links. The program product may also comprise data, databases or other information which may be accessible to, but not necessarily executed by, a processor.

Where a component (e.g. a controller, model, assembly, device, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. For example:
Systems 12, 112 may be implemented using various types of programmable controllers or processors. For example, systems 12, 112 may comprise a programmable computer, an embedded processor or the like. Systems 12, 112 may comprise more than one such processor. Systems 12, 112 may also include memory (not shown) which stores program information and the like.
In physical embodiments, various features of systems 12, 112 may reside in the same physical component. For example, in system 12 of FIG. 3, model 11 and light controller 10 are shown as being distinct features. However, those skilled in the art will appreciate that system 12 may comprise a single processor and that both model 11 and light controller 10 may be implemented on such a processor.
The systems and methods described above make use of the Jewett-Kronauer model equations (1)-(10B) which represent one particular model of the response of the human circadian cycle to light stimulus. Those skilled in the art will appreciate that other models may exist in the art or may become known in the future. The invention should be understood to include such alternative models. Such models may be used as a basis for any of the models of systems 12, 112. Those skilled in the art will also appreciate that such models are not limited to light stimulus, nor are they limited to a single type of stimulus.
Systems 12, 112 described above make use of light stimulus. Those skilled in the art will appreciate that the models used in systems 12, 112 may model the response of the human circadian cycle to different stimulus, such as melatonin stimulus, heat stimulus and audio stimulus for example. The light source(s) of systems 12, 112 may be replaced by one or more additional and/or alternative sources of stimuli.
Systems 12, 112 described above make use of light stimulus to modify a subject's circadian cycle. Those skilled in the art will appreciate that different wavelengths of light stimulus will have different effects on the circadian cycle. Those skilled in the art will also appreciate that this information may be incorporated into systems 12, 112 and/or into the models associated therewith. For example, the models used in systems 12, 122 may include light wavelength as a parameter, light sensors used in systems 12, 112 may sense a specific wavelength spectrum of light, and the light stimulus used in systems 12, 112 may be provided by one or more variable wavelength light sources.
The systems and methods of the invention are not limited to application on human subjects and may be used on animals given the appropriate model. For example, the circadian cycles of animals, such as racehorses, could also be modified to ensure peak performance at certain times.
Although light is a preferred stimulus, the invention may incorporate one or more other types of stimulus, including without limitation, sound, introduction of melatonin, introduction of food and the timing of physical activity.
Those skilled in the art will appreciate that there may be more than one circadian cycle in a subject, that there may be more than one circadian pacemaker, and that the invention may be applied to any combination of them.
   Accordingly, the scope of the invention is to be construed in accordance with the substance defined by the following claims.

1. A method for controllably adjusting a circadian cycle of a subject to track a reference trajectory, the method comprising:
providing at least one model representative of a response of a circadian state of the subject to a stimulus control signal; and
generating an optimal stimulus control signal using model predictive control based on the model,
wherein generating the optimal stimulus control signal comprises minimizing a cost function which comprises a cost term which is a function of a tracking error and wherein minimizing the cost function is subject to constraints which comprise minimum and maximum stimulus control signal levels.

2. A method according to claim 1 wherein the cost function comprises a cost term related to an amplitude of the stimulus control signal.

3. A method according to claim 2 wherein generating the optimal stimulus control signal is based, at least in part, on a circadian state feedback signal.

4. A method according to claim 3 wherein the stimulus control signal comprises a light control signal and wherein the method comprises applying the light control signal to determine an intensity of one or more light sources.

5. A method according to claim 4 wherein the at least one model comprises a response predictor subject model and wherein generating the optimal stimulus control signal comprises predicting a free response of the circadian state of the subject starting at a current time and extending out to a control horizon time using the response predictor subject model.

6. A method according to claim 5 wherein predicting the free response comprises assuming that a current value of the circadian state feedback signal represents an initial condition and that the light control signal remains constant between the current time and the control horizon time.

7. A method according to claim 6 wherein the response predictor subject model comprises a mathematical model defined by a plurality of differential equations.

8. A method according to claim 7 wherein the mathematical model is based on a Jewett-Kronauer model.

9. A method according to claim 8 wherein the mathematical model comprises a linearized version of the Jewett-Kronauer model.

10. A method according to claim 5 wherein the at least one model comprises a controller subject model and wherein generating the optimal stimulus control signal comprises determining an optimal series of control moves starting at the current time and extending out to the control horizon time using the controller subject model.

11. A method according to claim 10 wherein determining the optimal series of control moves is based, at least in part, on the free response of the circadian state of the subject, the reference trajectory and the circadian state feedback signal.

12. A method according to claim 11 wherein the controller subject model comprises a mathematical model defined by a plurality of differential equations.

13. A method according to claim 8 wherein the mathematical model is based on a Jewett-Kronauer model.

14. A method according to claim 13 wherein the mathematical model comprises a linearized version of the Jewett-Kronauer model.

15. A method according to claim 10 wherein the circadian state feedback signal is determined by modeling the response of the circadian state of the subject to the light control signal.

16. A method according to claim 15 modeling the response of the subject to the light control signal comprises using a mathematical model based on a Jewett-Kronauer model.

17. A method according to claim 16 wherein the mathematical model comprises a linearized version of the Jewett-Kronauer model.

18. A method according to claim 3 wherein the circadian state feedback signal is determined by modeling the response of the circadian state of the subject to the stimulus control signal.

19. A method according to claim 18 modeling the response of the subject to the stimulus control signal comprises using a mathematical model based on a Jewett-Kronauer model.

20. A method according to claim 19 wherein the mathematical model comprises a linearized version of the Jewett-Kronauer model.

21. A method according to claim 10 wherein the circadian state feedback signal is determined by estimating the circadian state of the subject based, at least in part, on one or more sensed parameters, the sensed parameters relating to the physiology of the subject.

22. A method according to claim 21 wherein the one or more sensed parameters include at least one of: heart rate, core body temperature, respiration, endocrine function levels, physical activity levels, blood pressure, blood oxygen concentration and skin temperature.

23. A method according to claim 3 wherein the circadian state feedback signal is determined by estimating the circadian state of the subject based, at least in part, on one or more sensed parameters, the sensed parameters relating to the physiology of the subject.

24. A method according to claim 23 wherein the one or more sensed parameters include at least one of: heart rate, core body temperature, respiration, endocrine function levels, physical activity levels, blood pressure, blood oxygen concentration and skin temperature.

25. A method according to claim 10 wherein predicting the free response is based, at least in part, on a light estimate signal representative of a light intensity experienced by the subject.

26. A method according to claim 25 wherein determining the optimal series of control moves is based, at least in part, on the light estimate signal.

27. A method according to claim 26 comprising estimating the light intensity experienced by the subject using a model to obtain the light estimate signal.

28. A method according to claim 26 comprising sensing the light intensity experienced by the subject using at least one light sensor to obtain the light estimate signal.

29. A method according to claim 13 wherein the stimulus control signal is provided in a domain of a driving input B of the Jewett-Kronauer model and wherein the method comprises converting the stimulus control signal from the domain of the driving input B to the light control signal in a light intensity domain I.

30. A method according to claim 29 comprising converting the minimum and maximum stimulus control signal levels from a light intensity domain I to a domain of a driving input B of the Jewett-Kronauer model.

31. A method according to claim 2 wherein the cost function comprises a first weighting factor associated with the tracking error cost term and a second weighting factor associated with the stimulus control signal cost term.

32. A method according to claim 31 wherein the first and second weighting factors vary over a duration of the circadian cycle of the subject.

33. A method for controllably adjusting a circadian cycle of a subject to track a reference trajectory, the method comprising:
obtaining input information, the input information comprising a signal representative of the reference trajectory, a feedback signal representative of a circadian state of the subject and one or more stimulus level constraints;
predicting a free response of the circadian state of the subject starting at a current time and extending out to a first future time using a response predictor subject model representative of a response of the circadian state of the subject to a stimulus;
determining an optimal series of control moves starting at the current time and extending out to second future time using a controller subject model representative of a response of the circadian state of the subject to a stimulus, the optimal series of control moves based, at least in part, on the free response of the circadian state of the subject, the reference trajectory signal and the current circadian state feedback signal; and
outputting a stimulus control signal to one or more stimulus sources, the stimulus control signal comprising a current one of the series of control moves; and
applying the stimulus control signal to one or more stimulus sources to determine an intensity thereof.

34. A method according to claim 33 wherein the response predictor subject model comprises a mathematical model based on a Jewett-Kronauer model.

35. A method according to claim 33 wherein the controller subject model comprises a mathematical model based on a linearized version of a Jewett-Kronauer model.

36. A system for controllably adjusting a circadian cycle of a subject to track a reference trajectory, the system comprising:
one or more inputs for receiving the reference trajectory and for receiving constraints which comprise minimum and maximum stimulus control signal levels;
a response predictor connected to receive a feedback signal representative of a circadian state of the subject, the response predictor configured to predict a free response of the circadian state of the subject starting at a current time and extending out to a first future time using a response predictor subject model representative of a response of the circadian state of the subject to a stimulus; and
a control sequence generator connected to receive the reference trajectory, the constraints, the feedback signal and the free response of the circadian state of the subject, the control sequence generator configured to determine an optimal series of control moves starting at the current time and extending out to a second future time using a controller subject model representative of a response of the circadian state of the subject to a stimulus, wherein the control sequence generator is connected to output a stimulus control signal to at least one stimulus source, the stimulus control signal comprising a current control move in the optimal series of control moves and the stimulus control signal determining an intensity of the at least one stimulus source.

37. A system according to claim 36 wherein the stimulus source comprises one or more light sources and the stimulus control signal comprises a light control signal representative of a light intensity of the one or more light sources.

38. A system according to claim 37 wherein the response predictor subject model comprises a mathematical model based on a Jewett-Kronauer model.

39. A system according to claim 37 wherein the controller subject model comprises a mathematical model based on a linearized version of a Jewett-Kronauer model.

40. A system according to claim 36 comprising one or more sensors for sensing one or more corresponding physiological parameters of the subject and a circadian state estimator which is connected to receive sensed values of the one or more physiological parameters from the one or more corresponding sensors and to estimate the feedback signal in response thereto.

41. A system according to claim 36 comprising a mathematical model which is connected to receive the stimulus control signal and which is configured to estimate the feedback signal in response thereto.

42. A system according to claim 37 comprising a converter for receiving the constraints which comprise minimum and maximum light control signal levels and converting the constraints from a light intensity domain I to a domain of a Jewett-Kronauer input signal B.

43. A system according to claim 42 comprising a converter for receiving the stimulus control signal in the domain of the Jewett-Kronauer input signal B and converting the stimulus control signal to the light control signal in the light intensity domain I.

44. A system for controllably adjusting a circadian cycle of a subject to track a reference trajectory, the system comprising:
one or more inputs for receiving the reference trajectory and for receiving constraints which comprise minimum and maximum stimulus control signal levels;
means for predicting a free response of a circadian state of the subject starting at a current time and extending out to a first future time based, at least in part, on a feedback signal representative of the circadian state of the subject;
means for determining an optimal series of control moves starting at the current time and extending out to a second future time based, at least in part, on the reference trajectory, the constraints, the feedback signal and the free response of the circadian state of the subject; and
means for applying a stimulus control signal to at least one stimulus source, the stimulus control signal comprising at least a portion of the optimal series of control moves and the stimulus control signal determining an intensity of the at least one stimulus source.

45. A method for altering a phase of a circadian cycle of a subject, the method comprising:
providing a controller subject model representing a response of a circadian state of the subject to a stimulus;
receiving a reference circadian trajectory, the reference circadian trajectory phase shifted from a current circadian cycle of the subject;
determining a series of stimulus control moves predicted by the controller subject model to result in the circadian cycle of the subject changing so as to track the reference circadian trajectory;
applying at least a portion of the series of stimulus control moves to one or more stimulus sources, the one or more stimulus sources providing stimulus which is received by the subject; wherein determining the series of stimulus control moves comprises applying an optimization process using the controller subject model.

46. A method according to claim 45 comprising using the controller subject model to predict future circadian states of the subject and minimizing a cost function based, at least in part, on differences between the predicted future circadian states of the subject and the reference circadian trajectory.

47. A method according to claim 46 comprising modeling a current state of the circadian cycle of the subject by supplying the stimulus control moves as inputs to a model and supplying the modeled current state as a feedback input to the controller subject model.

48. A method according to claim 45 wherein the one or more stimulus sources comprise one or more light sources.

49. A method according to claim 45 wherein the controller subject model comprises a mathematical model based on a Jewett/Kronauer model.
